(19) United States
(12) Patent Application Publication
(10) Pub. No.: US 2023/0248545 A1
(43) Pub. Date: Aug. 10, 2023

(54) BELOW-ELBOW PROSTHETIC ARM ASSEMBLY

(71) Applicant: SOUTHERN TAIWAN UNIVERSITY OF SCIENCE AND TECHNOLOGY, Tainan City (TW)

(72) Inventors: YANG-KUN OU, Tainan City (TW); CHIA-HSUAN LIAO, Tainan City (TW); HSUAN-YEN LEE, Tainan City (TW); KAI-JUNG PENG, Tainan City (TW); FENG-JIA ZHENG, Tainan City (TW); WEI MING KOK, Tainan City (TW); YU-KUAN MO, Tainan City (TW)

(21) Appl. No.: 17/892,151

(22) Filed: Aug. 22, 2022

(30) Foreign Application Priority Data
Feb. 9, 2022 (TW) 111104793

(51) Int. Cl. A61F 2/58

(52) U.S. Cl. CPC A61F 2/583; A61F 2002/543

(57) ABSTRACT

A below-elbow prosthetic arm assembly includes a hand; an arm connected to the hand; a cylinder connected to the arm; and a string connected to the rear end of the cylinder. To start using the prosthetic arm, the user's amputated upper limb is enclosed by the cylinder and tied thereto with a string. The below-elbow prosthetic arm assembly further includes an expansion kit adapted to meet the user's needs in daily lives, learning and sports. The expansion kit is directly fitted to the hand. Function expansion is achieved without removing the hand. Therefore, the below-elbow prosthetic arm exhibits enhanced ease of use and minimizes the user's financial burdens which might otherwise arise from ordering customized prosthetic arms for use in different activities.

### CROSS-REFERENCE TO RELATED APPLICATION

This non-provisional application claims priority under 35 U.S.C. § 119(a) on Patent Application No(s). 111104793 filed in Taiwan, R.O.C. on Feb. 9, 2022, the entire contents of which are hereby incorporated by reference.

### BACKGROUND OF THE INVENTION

### 1. Field of the Invention

The present disclosure relates to a below-elbow prosthetic arm assembly, and in particular to a prosthetic arm with an expansion kit replaceable to meet user needs in daily lives, learning and sports and facilitate function expansion.

### 2. Description of the Related Art

Some persons who have an accident or disease may need amputation. After receiving therapy, the amputees usually have to wear prostheses in order to maintain the quality of their daily lives. The amputees, especially growing pediatric amputees, need customized prostheses in accordance with specific growth stages.

However, designers of existing children's prostheses give no considerations to changes in the size of amputated limbs and user needs in daily lives in terms of dining, learning and sports. As a result, growing pediatric amputees have to change prostheses very often to the detriment of environmental protection, not to mention adding to their parents' financial burdens and causing a waste of material resources.

Therefore, in view of the aforesaid drawbacks of conventional children's prostheses, there is still room for improvement in the prior art.

### BRIEF SUMMARY OF THE INVENTION

The disclosure relates to a below-elbow prosthetic arm assembly. It is an objective of the disclosure to provide a prosthetic arm with an expansion kit replaceable to meet user needs in daily lives, learning and sports and facilitate function expansion.

In order to achieve the above and other objectives, the disclosure provides an below-elbow prosthetic arm assembly, comprising: a hand; an arm connected to a rear end of the hand; a cylinder connected to a rear end of the arm; a holder connected to a rear end of the cylinder and to a string; and at least one expansion kit comprising a hand fitting portion and a function expansion portion, with the hand fitting portion being fitted to the hand.

Regarding the below-elbow prosthetic arm assembly, the expansion kit comprises a dining component, and the function expansion portion of the dining component is a base dented to form a bowl chamber.

Regarding the below-elbow prosthetic arm assembly, a friction layer is coupled to an inner surface of the bowl chamber of the expansion kit.

Regarding the below-elbow prosthetic arm assembly, the expansion kit comprises a paperweight component, and the function expansion portion of the paperweight component is a block, with a flat surface being formed at a bottom of the block.

Regarding the below-elbow prosthetic arm assembly, a soft pad is coupled to the flat surface at the bottom of the block.

Regarding the below-elbow prosthetic arm assembly, the expansion kit comprises a sports component, and the function expansion portion of the sports component is a sleeve that tilts.

Regarding the below-elbow prosthetic arm assembly, a shaft is connected to a rear end of the hand and fitted to a positioning disk, with first slots being formed at a circumferential edge of the positioning disk, and the arm has therein a hollow-core chamber, with a through hole being disposed at a front end of the arm and being in communication with the chamber, wherein the shaft connected to the hand is penetratingly disposed at the through hole disposed at the front end of the arm, with the positioning disk being disposed in the chamber, with a first tenon being formed on the wall of the chamber to engage with any one first slot of the positioning disk.

Regarding the below-elbow prosthetic arm assembly, the arm has therein a hollow-core chamber, with a through hole being disposed at the rear end of the arm and being in communication with the chamber, with the chamber having a cylinder fitting portion positioned proximate to the through hole at the rear end of the arm, with a polygonal fitting hole being disposed at a center of the cylinder fitting portion, with at least one penetrating hole penetrating a sidewall of the arm and thus being in communication with the polygonal fitting hole, with a polygonal fitting block being disposed at a front end of the cylinder, with at least one fastening hole being disposed on a lateral side of the polygonal fitting block to allow the front end of the cylinder to be inserted into the through hole at the rear end of the arm and fitted inside the chamber of the arm, wherein the polygonal fitting block disposed at the front end of the cylinder is fitted to the polygonal fitting hole of the arm, and the fastening hole disposed on the lateral side of the polygonal fitting block corresponds in position to the penetrating hole of the arm, with at least one fastening element being penetratingly disposed at the penetrating hole of the arm and fastened to the fastening hole of the polygonal fitting block of the cylinder.

Regarding the below-elbow prosthetic arm assembly, connection ribs are disposed between the holder and the cylinder and each have two ends coupled to the holder and the cylinder, respectively.

Regarding the below-elbow prosthetic arm assembly, an axial hole is disposed on a side of the holder, and second slots are formed in the wall of the axial hole, wherein second tenons are formed at the bottom of a coiling drum to engage with the second slots of the axial hole, and the coiling drum is connected to an axle, wherein the holder is covered with a cover and coupled thereto to hide the coiling drum, the cover having a rotation hole, the axle being penetratingly disposed at the rotation hole, wherein a knob is disposed on an outer side of the cover and fitted to the axle, wherein an end of a cord is fitted to the coiling drum, and another end of the cord is inserted into the arm and connected to branch cords, wherein the hand has a palm connected to fingers, and the branch cords are penetratingly connected to the fingers of the hand, respectively.

To start using the prosthetic arm, the user's amputated upper limb is enclosed by the cylinder and tied thereto with a string. The below-elbow prosthetic arm assembly further includes an expansion kit adapted to meet the user's needs in daily lives, learning and sports. The expansion kit is directly fitted to the hand. Function expansion is achieved without removing the hand. Therefore, the below-elbow prosthetic arm exhibits enhanced ease of use and minimizes the user's financial burdens which might otherwise arise from ordering customized prosthetic arms for use in different activities.

### BRIEF DESCRIPTION OF THE DRAWINGS

FIG. 1 is an exploded view of a below-elbow prosthetic arm assembly of the disclosure.

FIG. 2 is a perspective view of the below-elbow prosthetic arm assembly of the disclosure.

FIG. 3 is another perspective view of the below-elbow prosthetic arm assembly of the disclosure.

FIG. 4 is a cross-sectional view of the below-elbow prosthetic arm assembly of the disclosure.

FIG. 5 is a partial enlarged cross-sectional view of the below-elbow prosthetic arm assembly of the disclosure.

FIG. 6 is another partial enlarged cross-sectional view of the below-elbow prosthetic arm assembly of the disclosure.

FIG. 7 is a partial enlarged top view of the below-elbow prosthetic arm assembly of the disclosure.

FIG. 8 is an exploded view of an expansion kit of the disclosure.

FIG. 9 is a first perspective view of the expansion kit of the disclosure.

FIG. 10 is a second perspective view of the expansion kit of the disclosure.

FIG. 11 is a third perspective view of the expansion kit of the disclosure.

### DETAILED DESCRIPTION OF THE INVENTION

Features and advantages of the disclosure are hereunder completely and clearly illustrated with specific embodiments, depicted with drawings, and described below.

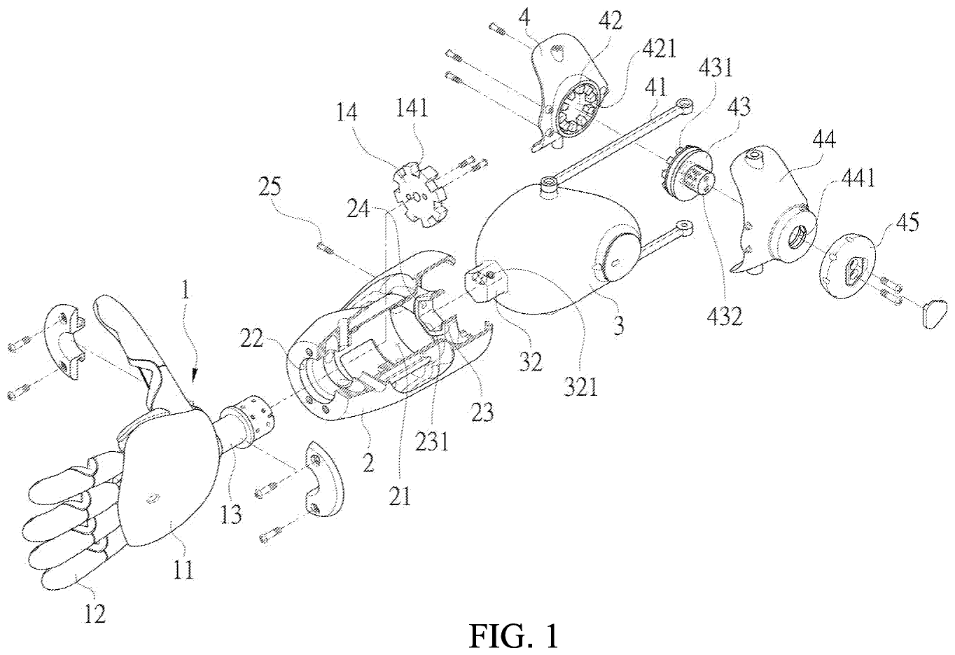

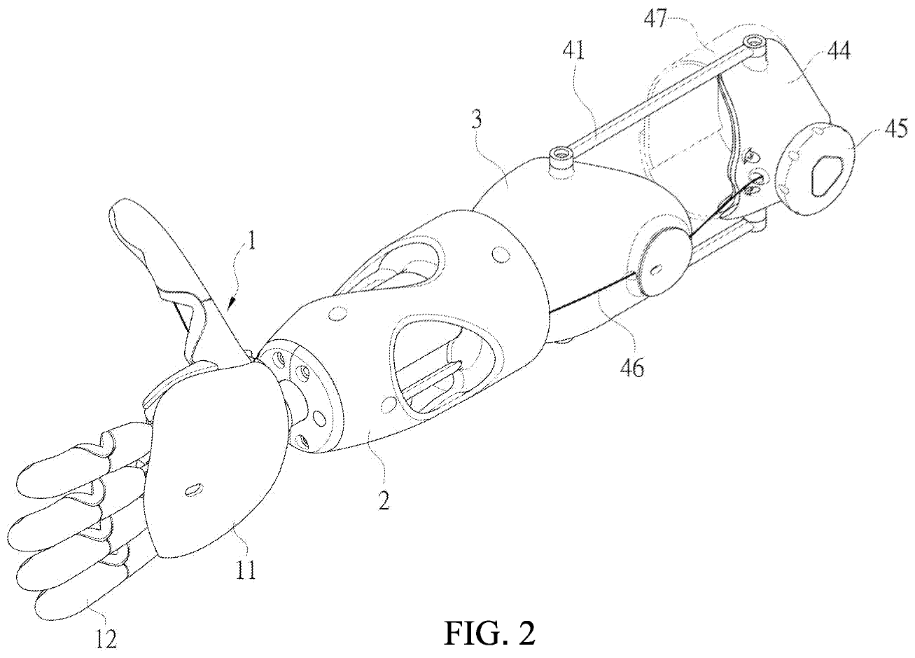

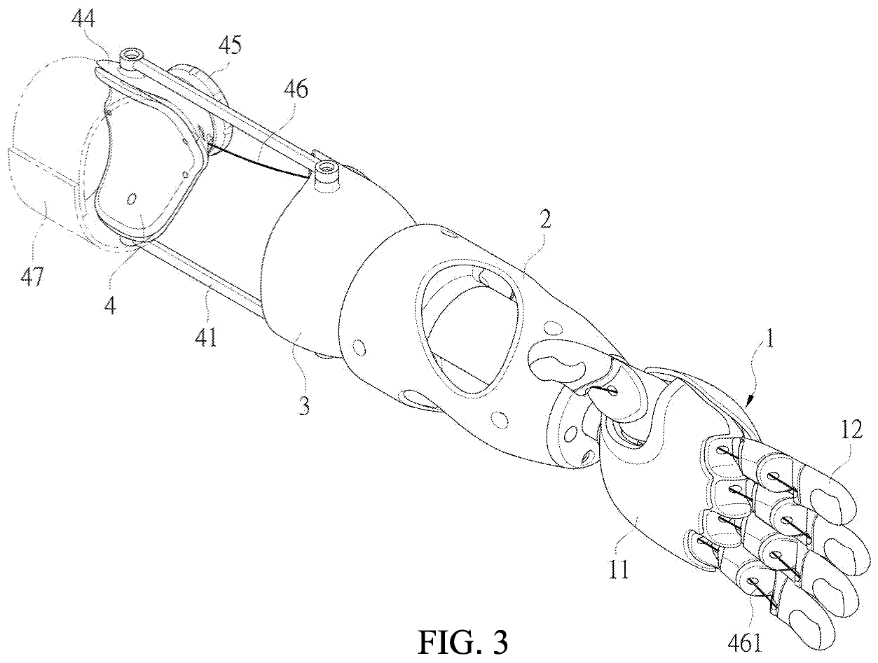

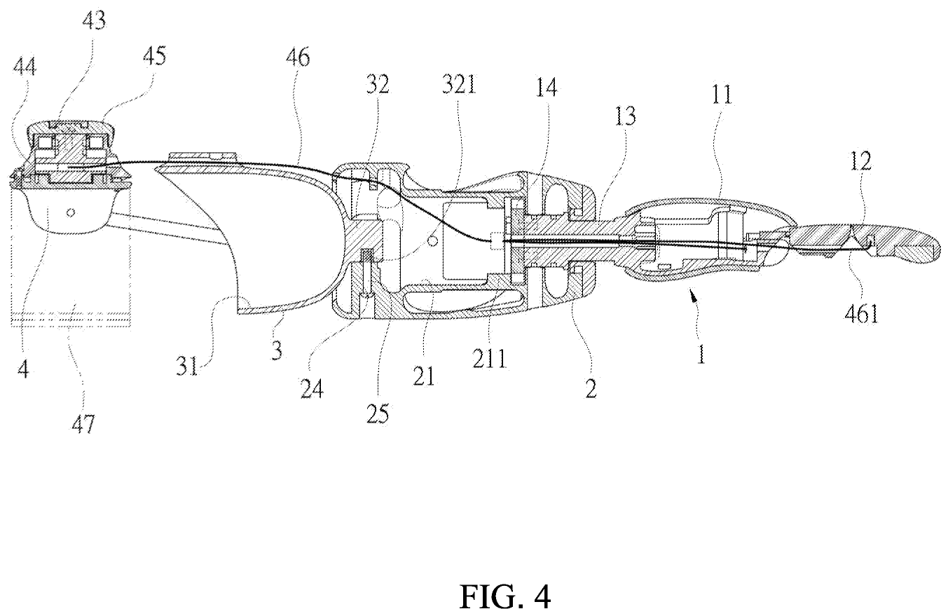

Referring to FIGS. 1-4, a below-elbow prosthetic arm assembly of the disclosure essentially comprises a hand (1), an arm (2), a cylinder (3), a holder (4), and at least one expansion kit (5).

The hand (1) comprises a palm (11). Fingers (12) are connected to the palm (11). A shaft (13) is connected to the rear end of the palm (11). The shaft (13) is fitted to a positioning disk (14). First slots (141) formed at the circumferential edge of the positioning disk (14).

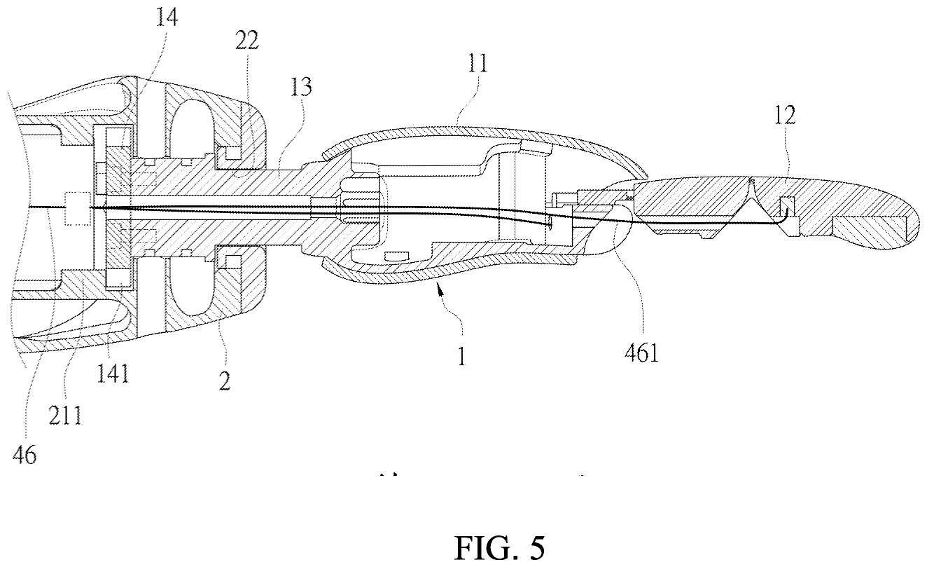

The arm (2) has therein a hollow-core chamber (21). Two through holes (22) are disposed at the front end and rear end of the arm (2), respectively. The through holes (22) are in communication with the chamber (21). The shaft (13) of the hand (1) is penetratingly disposed at the through hole (22) at the front end of the arm (2), and the positioning disk (14) is disposed in the chamber (21). Referring to FIG. 5, a first tenon (211) is formed on the wall of the chamber (21) to engage with any one first slot (141) of the positioning disk (14). The chamber (21) has a cylinder fitting portion (23) positioned proximate to the through hole (22) at the rear end of the arm (2). A polygonal fitting hole (231) is disposed at the center of the cylinder fitting portion (23). At least one penetrating hole (24) penetrates the sidewall of the arm (2) and thus is in communication with the polygonal fitting hole (231).

The cylinder (3) has therein a socket (31). A polygonal fitting block (32) is disposed at the front end of the cylinder (3). At least one fastening hole (321) is disposed on a lateral side of the polygonal fitting block (32) to allow the front end of the cylinder (3) to be inserted into the through hole (22) at the rear end of the arm (2) and fitted inside the chamber (21) of the arm (2). The polygonal fitting block (32) disposed at the front end of the cylinder (3) is fitted to the polygonal fitting hole (231) disposed in the chamber (21) of the arm (2). The fastening hole (321) disposed on the lateral side of the polygonal fitting block (32) corresponds in position to the penetrating hole (24) disposed on the sidewall of the arm (2). At least one fastening element (25) is penetratingly disposed at the penetrating hole (24) of the arm (2) and fastened to the fastening hole (321) of the polygonal fitting block (32) of the cylinder (3).

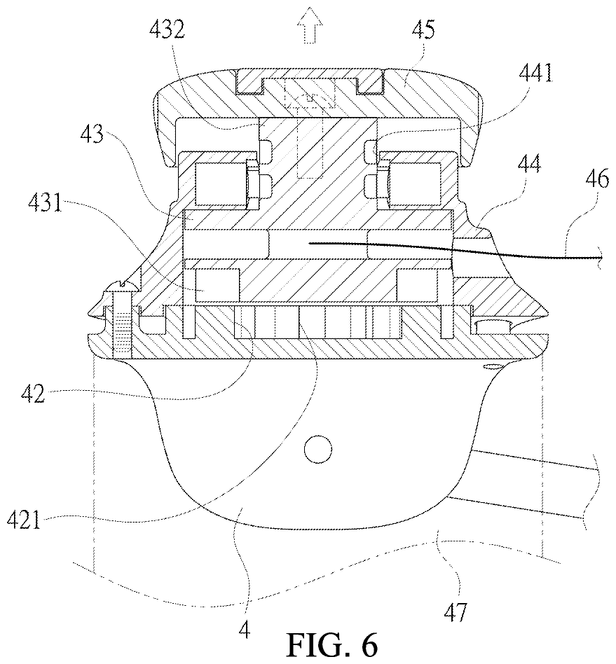

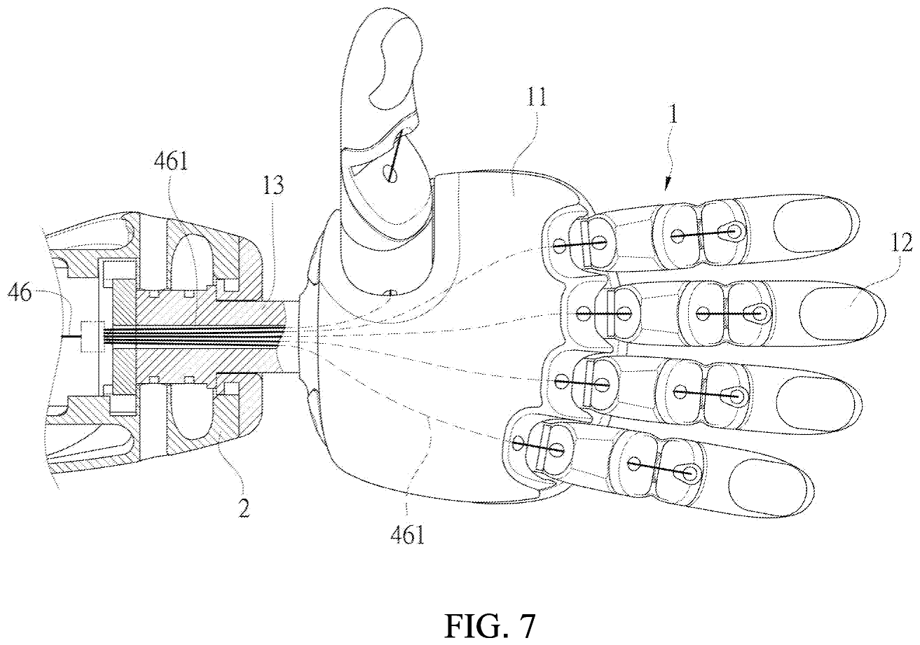

The holder (4) is disposed behind the cylinder (3), spaced apart therefrom, and connected to the cylinder (3) by connection ribs (41). An axial hole (42) is disposed on one side of the holder (4). Second slots (421) are formed in the wall of the axial hole (42). Referring to FIG. 6, a coiling drum (43) is provided. Second tenons (431) are formed at the bottom of the coiling drum (43) to engage with the second slots (421) of the axial hole (42). The coiling drum (43) is connected to an axle (432). The holder (4) is covered with a cover (44) and coupled thereto to hide the coiling drum (43). The cover (44) has a rotation hole (441). The axle (432) is penetratingly disposed at the rotation hole (441). A knob (45) is disposed on the outer side of the cover (44) and fitted to the axle (432). One end of a cord (46) is fitted to the coiling drum (43), and the other end of the cord (46) is inserted into the chamber (21) of the arm (2). Referring to FIG. 7, the other end of the cord (46) is connected to branch cords (461), and the branch cords (461) are penetratingly connected to the fingers (12) of the hand (1), respectively. As shown in FIG. 7, a string (47) is connected to the holder (4), and the two ends of the string (47) each have an engaging element, such as Velcro tape.

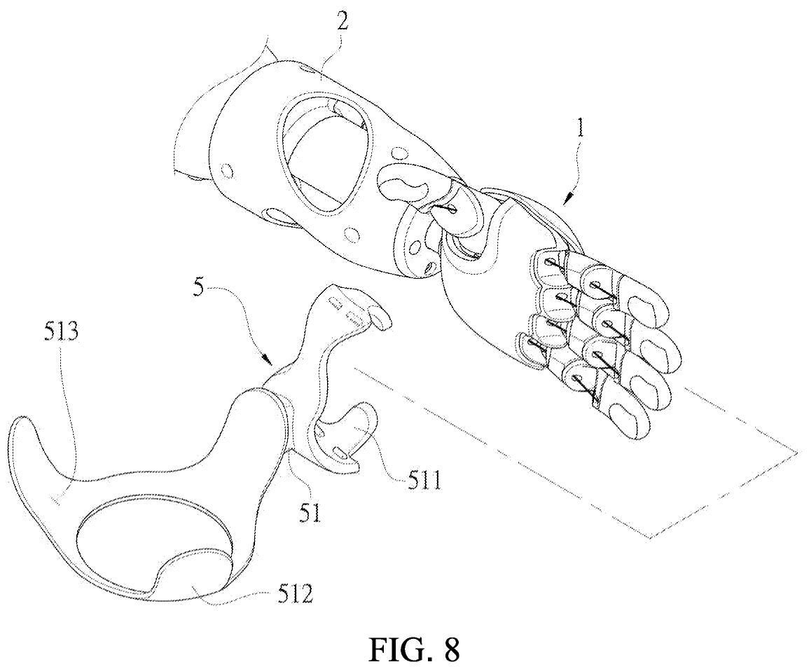

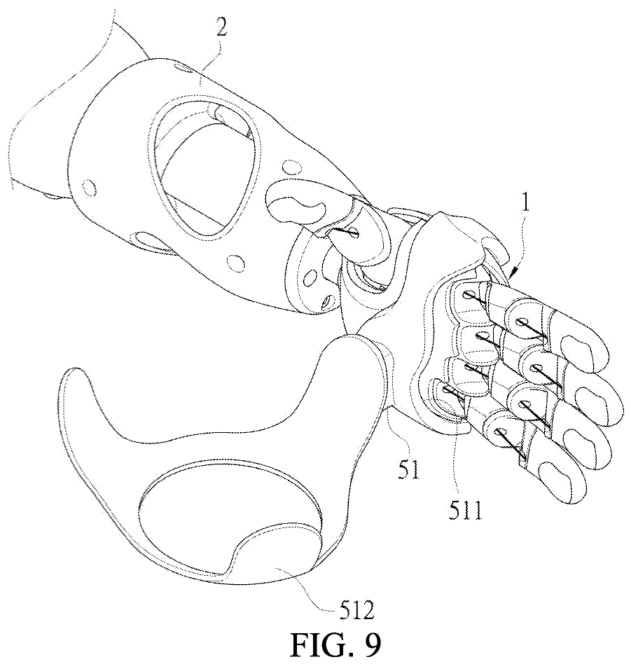

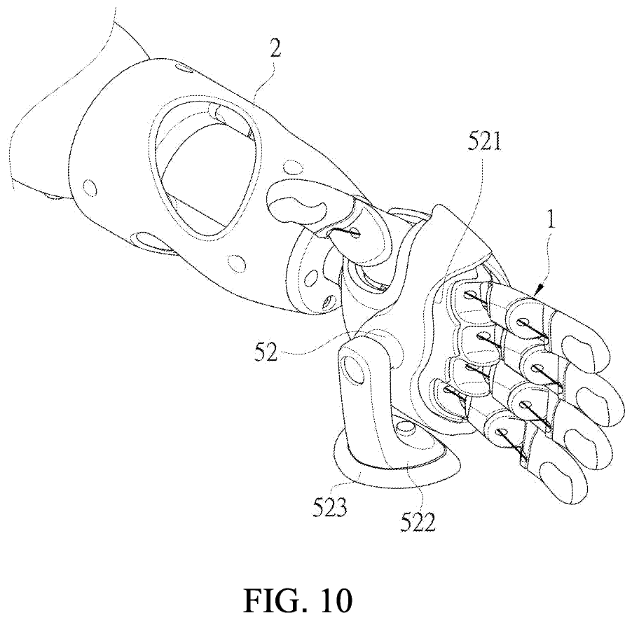

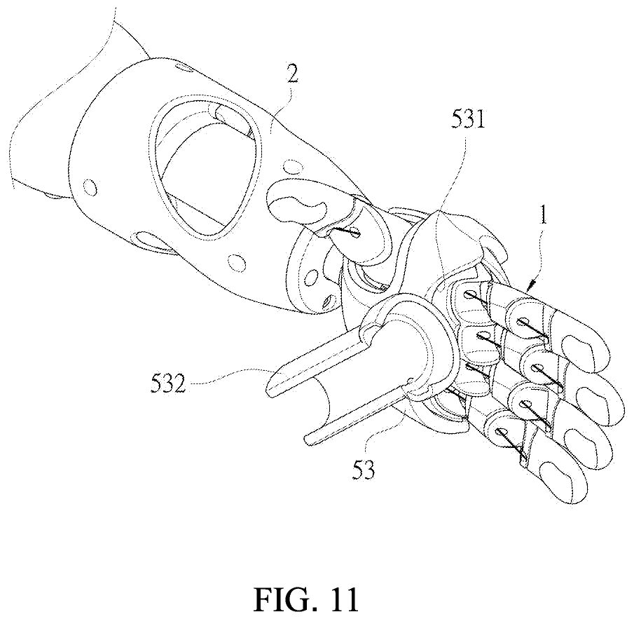

Referring to FIGS. 8-11, the at least one expansion kit (5) comprises a dining component (51) (shown in FIGS. 8, 9), a paperweight component (52) (shown in FIG. 10), or a sports component (53) (shown in FIG. 11). The dining component (51), paperweight component (52) and sports component (53) have C-shaped hand fitting portions (511), (521), (531), respectively, in order to be fitted to the hand (1). The dining component (51) has a function expansion portion (512) which is a base dented to form a bowl chamber (513). A silicone friction layer is coupled to the inner surface of the bowl chamber (513). The paperweight component (52) has a function expansion portion (522) which is a block. A flat surface is formed at the bottom of the block. A soft pad (523) is coupled to the flat surface at the bottom of the block. The sports component (53) has a function expansion portion (532) which is a sleeve that tilts.

To mount the prosthetic arm of the disclosure on a user's amputated upper limb, the holder (4) is tied to the upper arm of the user's amputated upper limb with the string (47), and then the tip of the amputated upper limb is fitted into the socket (31) of the cylinder (3). Then, the knob (45) at the holder (4) is pulled outward, such that the second tenons (431) disposed at the bottom of the coiling drum (43) connected to the knob (45) are disengaged from the second slots (421) disposed in the wall of the axial hole (42) of the holder (4); at this point in time, the knob (45) is rotated to drive the coiling drum (43) to take in or let out the cord (46) and thereby adjust the tightness thereof. After the tightness of the cord (46) has been appropriately adjusted, the knob (45) can be pushed inward to reset the coiling drum (43) connected to the knob (45), and in consequence the second tenons (431) of the coiling drum (43) get engaged with the second slots (421) of the axial hole (42) again. Thus, by flexing his or her arm, the user can flex the fingers (12) of the hand (1) connected to the cord (46) to, for example, grab an object.

To rotate the hand (1) slightly, it is feasible to put the hand (1) outward by a short distance, such that the first slots (141) of the positioning disk (14) connected to the hand (1) are disengaged from the first tenon (211) disposed on the inner wall of the chamber (21) of the arm (2). After the hand (1) has been rotated by a desirable angle, the hand (1) is pushed inward to reset the positioning disk (14) connected to the hand (1); thus, the first slots (141) of the positioning disk (14) get engaged with the first tenon (211) disposed at the arm (2) again. Both the cylinder (3) and the holder (4) are manufactured by 3D printing and thus can be customized according to the shape of an amputee's amputated upper limb and able to enclose it well and firmly.

The disclosure is further advantageous in that the cylinder (3) and the arm (2) can be movably removed, such that a custom-made cylinder (3) and a custom-made holder (4) can be ordered in case of a change in the size of the growing pediatric amputee's amputated upper limb. Therefore, the disclosure dispenses with the need to change a prosthesis entirely and thus minimizes the growing pediatric amputee's parents' financial burdens.

The expansion kit (5) comprises the dining component (51), paperweight component (52) and sports component (53). The dining component (51), paperweight component (52) and sports component (53) have the C-shaped hand fitting portions (511), (521), (531), respectively, in order to be movably fitted to the hand (1). Thus, the expansion kit (5) is replaceable to not only meet user needs in daily lives, learning and sports but also achieve function expansion without removing the hand (1). The bowl chamber (513) and the function expansion portion (512) enable the dining component (51) to contain a dining bowl and assist the amputee in holding the dining bowl. The silicone friction layer disposed on the inner surface of the bowl chamber (513) augments the friction required for fixing the dining bowl in place and prevents the dining bowl from toppling or inappropriately rotating. The flat surface disposed at the bottom of the block-shaped function expansion portion (522) enables the paperweight component (52) to press against a sheet of paper and assists the amputee in writing on the paper. The sports component (53) has the function expansion portion (532) which is sleeve-shaped and tilts; thus, a handle of a jump rope can be fitted into the sports component (53) and grabbed by hand, allowing the amputee to perform rope jumping. Thus, the disclosure dispenses with the need to place orders for customized, dedicated prostheses for use in respective activities, so as to minimize the growing pediatric amputee's parents' financial burdens and prevent a waste of material resources.

The above embodiments and drawings are not restrictive of the below-elbow prosthetic arm assembly of the disclosure. Thus, appropriate modifications and changes made by persons skilled in the art to the embodiments without departing from the scope of the disclosure shall be deemed falling within the scope of the claims of the disclosure.

In conclusion, the embodiments of the disclosure achieve anticipated benefits. Specific structures disclosed in the embodiments are neither adopted by similar existing products nor published.

What is claimed is:

1. An below-elbow prosthetic arm assembly, comprising:
a hand;
an arm connected to a rear end of the hand;
a cylinder connected to a rear end of the arm;
a holder connected to a rear end of the cylinder and to a string; and
at least one expansion kit comprising a hand fitting portion and a function expansion portion, with the hand fitting portion being fitted to the hand.

2. The below-elbow prosthetic arm assembly of claim 1, wherein the expansion kit comprises a dining component, and the function expansion portion of the dining component is a base dented to form a bowl chamber.

3. The below-elbow prosthetic arm assembly of claim 2, wherein a friction layer is coupled to an inner surface of the bowl chamber of the expansion kit.

4. The below-elbow prosthetic arm assembly of claim 1, wherein the expansion kit comprises a paperweight component, and the function expansion portion of the paperweight component is a block, with a flat surface being formed at a bottom of the block.

5. The below-elbow prosthetic arm assembly of claim 4, wherein a soft pad is coupled to the flat surface at the bottom of the block.

6. The below-elbow prosthetic arm assembly of claim 1, wherein the expansion kit comprises a sports component, and the function expansion portion of the sports component is a sleeve that tilts.

7. The below-elbow prosthetic arm assembly of claim 1, wherein a shaft is connected to a rear end of the hand and fitted to a positioning disk, with first slots being formed at a circumferential edge of the positioning disk, and the arm has therein a hollow-core chamber, with a through hole being disposed at a front end of the arm and being in communication with the chamber, wherein the shaft connected to the hand is penetratingly disposed at the through hole disposed at the front end of the arm, with the positioning disk being disposed in the chamber, with a first tenon being formed on the wall of the chamber to engage with any one first slot of the positioning disk.

8. The below-elbow prosthetic arm assembly of claim 1, wherein the arm has therein a hollow-core chamber, with a through hole being disposed at the rear end of the arm and being in communication with the chamber, with the chamber having a cylinder fitting portion positioned proximate to the through hole at the rear end of the arm, with a polygonal fitting hole being disposed at a center of the cylinder fitting portion, with at least one penetrating hole penetrating a sidewall of the arm and thus being in communication with the polygonal fitting hole, with a polygonal fitting block being disposed at a front end of the cylinder, with at least one fastening hole being disposed on a lateral side of the polygonal fitting block to allow the front end of the cylinder to be inserted into the through hole at the rear end of the arm and fitted inside the chamber of the arm, wherein the polygonal fitting block disposed at the front end of the cylinder is fitted to the polygonal fitting hole of the arm, and the fastening hole disposed on the lateral side of the polygonal fitting block corresponds in position to the penetrating hole of the arm, with at least one fastening element being penetratingly disposed at the penetrating hole of the arm and fastened to the fastening hole of the polygonal fitting block of the cylinder.

9. The below-elbow prosthetic arm assembly of claim 1, wherein connection ribs are disposed between the holder and the cylinder and each have two ends coupled to the holder and the cylinder, respectively.

10. The below-elbow prosthetic arm assembly of claim 1, wherein an axial hole is disposed on a side of the holder, and second slots are formed in the wall of the axial hole, wherein second tenons are formed at the bottom of a coiling drum to engage with the second slots of the axial hole, and the coiling drum is connected to an axle, wherein the holder is covered with a cover and coupled thereto to hide the coiling drum, the cover having a rotation hole, the axle being penetratingly disposed at the rotation hole, wherein a knob is disposed on an outer side of the cover and fitted to the axle, wherein an end of a cord is fitted to the coiling drum, and another end of the cord is inserted into the arm and connected to branch cords, wherein the hand has a palm connected to fingers, and the branch cords are penetratingly connected to the fingers of the hand, respectively.
